(19) United States
(12) Patent Application Publication
(10) Pub. No.: US 2019/0111260 A1
(43) Pub. Date: Apr. 18, 2019

(54) Optical Proximity Sensing System For Atraumatic Cochlea Implant Surgery

(71) Applicants: ECOLE POLYTECHNIQUE FEDERALE DE LAUSANNE (EPFL), Lausanne (CH); Massachusetts Eye and Ear Infirmary, Boston, MA (US)

(72) Inventors: Demetri Psaltis, Lausanne (CH); Ye Pu, Préverenges (CH); Christophe Moser, Lausanne (CH); Konstantina Stankovic, Boston, MA (US)

(21) Appl. No.: 15/766,009

(22) PCT Filed: Oct. 5, 2016
(86) PCT No.: PCT/IB2016/055959

§ 371 Date: Apr. 5, 2018

Related U.S. Application Data
(60) Provisional application No. 62/237,629, filed on Oct. 6, 2015.

(51) Int. Cl. A61N 1/36; A61B 5/00

(52) U.S. Cl. CPC A61N 1/36036; A61B 5/6867; A61B 5/1072; A61B 5/6817; A61B 5/0084

(57) ABSTRACT

The design of a proximity sensor to be integrated into cochlea implants is described. The sensor allows the anticipation of contact between the cochlear implant and intracochlear structures, including the cochlear canal wall and basilar membrane, providing a feedback or an alarm to the surgeon performing the implant insertion such that trauma to the cochlea is avoided. This helps to preserve any residual hearing ability in patients who receive the surgical implant.

### FIELD OF THE INVENTION

The present invention relates to the procedure of surgically inserting a cochlear implant in human patients. In particular, this invention pertains to optical proximity sensing and surface profilometry in cochlear implants.

### BACKGROUND

Cochlea implants are devices that generate hearing sensation through electrical stimulation of the auditory sensory neurons using an array of electrodes in patients with partial or total hearing loss. FIG. 1 illustrates a placement of a cochlea implant, including a cochlear canal 101, a cochlear implant electrode array 102, sensory neurons 103, a malleus 104, an incus 106, stapes 104, an external ear canal 107, and ear drum 108, a microphone 109, a transmitter 110, and a receiver/stimulator 111. As shown in FIG. 1, the cochlear implant electrode array 102 is surgically inserted manually into one of the three spiral cochlear canal spaces 101, i.e., the scala tympani 201 shown in FIG. 2, where the electrodes 202 stimulate the spiral ganglion neurons 208. FIG. 2 shows a cross-sectional view of the cochlear canal with the implant electrode array cut at the dashed line in FIG. 1, including the scala tympani 201, the cochlear implant electrode array 202, a basilar membrane 203, an organ of Corti 204, a scala media 205, a scala vestibule 206, Reissner's membrane 207, and a spiral ganglion 208. However, during surgical insertion, no feedback is available to monitor the relative position of the implant as the surgeon maneuvers it in the tightly curled cochlear canal. Often, significant damage to the inner ear can occur due to misplacements of the implant as shown in the examples of FIGS. 3-6, resulting in loss of residual hearing ability. FIG. 3 shows a trauma induced by cochlear implant electrode array scrapping, and illustrates a scala tympani 301 and cochlear implant electrode array 302. FIG. 4 shows a trauma induced by folding of the cochlear implant electrode array, and illustrates the scala tympani 401 and cochlear implant electrode array 402. FIG. 5 shows a trauma induced by cochlear implant electrode array buckling, and illustrates the scala tympani 501 and cochlear implant electrode array 502. FIG. 6 shows a trauma induced by cochlear implant electrode array breaching the basilar membrane, and illustrates the scala tympani 601 and cochlear implant electrode array 602.

Today, technological advancements in cochlear implants have enabled implantation in patients with some degree of residual hearing. Recent studies have shown that preserving residual hearing is crucial for a significantly improved hearing performance through the simultaneous use of an electrical hearing, via a cochlear implant, and an acoustic hearing, via a hearing aid in the same ear. An atraumatic insertion that preserves the residual hearing ability is thus critical to reach the goal of a combined electric-acoustic hearing. Although some intracochlear damage can be avoided by the use of a short implant that does not reach beyond the basal turn of the cochlear canal, most people benefit from a deep insertion of a cochlear implant to allow stimulation of a wide range of frequencies. To achieve this goal, implants incorporating actuators or sensors have been invented, where a compact high-resolution sensor is of overwhelming importance.

One goal of the present invention is to provide an optical mechanism to be incorporated in cochlear implants to help surgeons guide the implant into the cochlea with controlled proximity to the modiolus and to minimize damage to intracochlear structures, including hair cells and neurons. The optical guidance is expected to enhance the efficacy of the implant by preserving any partial hearing capability for a combined electric-acoustic hearing. Furthermore, this optical mechanism can provide valuable information about the status of the hair cells and neurons in the cochlea, which can be used to optimize the controls of the implant and hearing aid.

### SUMMARY OF THE INVENTION

In a first aspect the invention provides a cochlear implant device comprising an implant body being delimited at least by an implant surface, an electrode array, and at least one proximity sensor. The at least one proximity sensor is configured to provide distance or contact information that is representative of a distance lying between the implant surface and any one of cochlear intra-canal structures.

In a preferred embodiment, the at least one proximity sensor comprises at least one optical waveguide and a photodetector, the at least one optical waveguide being configured to deliver light from a source to a specific location of the implant surface, the at least one optical waveguide being connected to the source, wherein the light is intended to impinge the cochlea intra-canal structures and to be scattered back to the implant surface. The at least one proximity sensor comprises a second optical waveguide being configured to collect the scattered light and deliver it to the photodetector. A signal at an output from the photodetector is configured to provide the distance or contact information.

In a further preferred embodiment, the at least one optical waveguide connected to the source and the second waveguide connected to the detector are a same optical waveguide, namely the at least one optical waveguide.

In a further preferred embodiment, the waveguide is a thin optical fiber.

In a further preferred embodiment, the proximity sensor comprises a plurality of optical waveguides, whereby at least one of the plurality of waveguides is configured to deliver light of at least one wavelength, and further such that the delivered light impinges and is scattered from the cochlear intra-canal structures, wherein scattered light is collected by the plurality of waveguides, the implant device further comprising processing means configured to process an optical power in each waveguide collected and to retrieve the distance information at the position of the sensor.

In a further preferred embodiment, the plurality of sensors is configured to form a canal surface profilometer.

In a further preferred embodiment, the proximity sensor comprises at least one optical waveguide configured to deliver light of multiple wavelengths from a source to a plurality of distinct locations on the implant surface in a one-to-one mapping through fiber Bragg gratings, where the light impinges the cochlear intra-canal structures and is scattered back to the implant surface, the at least one waveguide collecting said scattered light through the fiber Bragg gratings and is configured for an intended delivery of the scattered light to a spectral analyzer, wherein the optical power in each wavelength channel from said spectral analyzer provides distance information between the implant surface to the cochlear intra-canal structures at the position respective to the wavelength.

In a further preferred embodiment, the proximity sensor comprises at least one semiconductor microchip arranged at a specific position on the implant surface with necessary metal wirings for power and communications, the semiconductor microchip comprising at least one light source and one photodetector, the semiconductor microchip being configured such that light from the light source impinges the cochlear intra-canal structures and is scattered back to the implant surface, and the photodetector converts the light intensity into the distance information at the position of the sensor.

In a further preferred embodiment, the light source is a light emitting diode.

In a further preferred embodiment, the light source is a laser diode.

In a further preferred embodiment, the proximity sensor comprises a plurality of semiconductor microchips arranged at specific positions on the implant surface with necessary metal wirings for power and communications, each of the semiconductor microchips comprising at least one light source and one photodetector, light from the light source impinging the cochlear intra-canal structures and being scattered back to the implant surface, the photodetector converting the light intensity into the distance information at the position of that sensor.

In a further preferred embodiment, the light source is a light emitting diode.

In a further preferred embodiment, the light source is a laser diode.

In a further preferred embodiment, the plurality of sensors forms a canal surface profilometer.

In a second aspect, the invention provides a method for fabricating an optical waveguide for use in the implant device described herein above, wherein the optical waveguide is fabricated by an exposure of a waveguide material to a focused laser light such that the waveguide material exposed to the focused laser light obtains a different refractive index than the unexposed material, hence forming a waveguide.

In a further preferred embodiment of the method, the waveguide material comprises silica or polydiphenylsiloxane.

### BRIEF DESCRIPTION OF THE DRAWINGS

The invention will be understood through the detailed description of preferred embodiments and in reference to the appended drawings, wherein

FIG. 1 shows a placement of cochlea implant according to prior art;

FIG. 2 shows a cross-sectional view of the cochlear canal with the implant electrode array cut at the dashed line in FIG. 1;

FIG. 3 shows a trauma induced by cochlear implant electrode array scrapping;

FIG. 4 shows a trauma induced by folding of the cochlear implant electrode array;

FIG. 5 shows a trauma induced by cochlear implant electrode array buckling;

FIG. 6 shows a trauma induced by cochlear implant electrode array breaching the basilar membrane;

FIG. 7 shows a cochlear implant electrode array with proximity sensing according to an example embodiment of the invention;

FIG. 8 shows a waveguide proximity sensor according to an example embodiment of the invention;

FIG. 9 shows a semiconductor proximity sensor according to an example embodiment of the invention; and

FIG. 10 contains an operation diagram of atraumatic cochlea implant surgery.

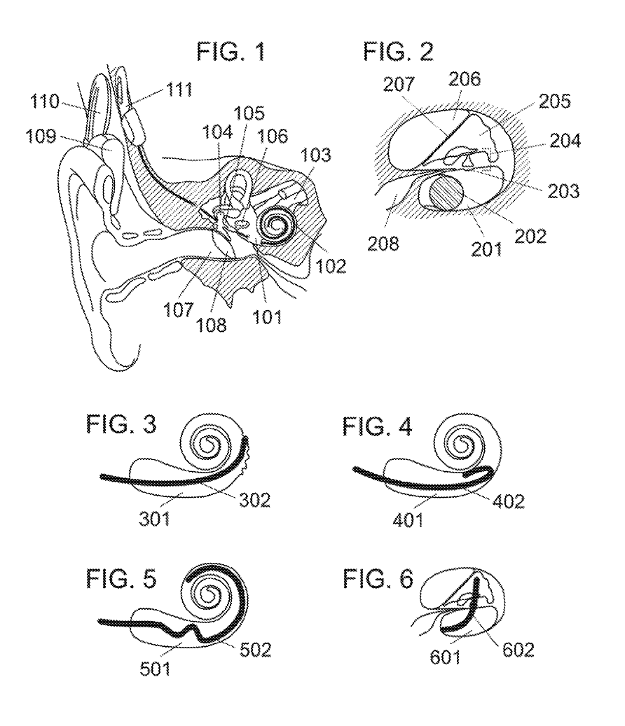

### DETAILED DESCRIPTION

The present invention concerns an optical proximity sensor based on optical waveguides or optoelectronic semiconductor microchips that are integrated into the cochlea implants. In the waveguide-type sensor, the waveguides can be realized either by changing the optical properties of the implant material or embedding foreign materials of proper optical properties. The distal end of the waveguide leads the incident light to a specific position of the implant surface, such that an increased light is returned to the proximal end when the surface approaches the intra-cochlear canal wall or basilar membrane. In the semiconductor-type sensor, optoelectronic microchips are embedded into the implant body at specific locations, which draws power and communicates with the outside through metal wirings. Each microchip contains a semiconductor light source and a photodetector, such that an increased light is returned to the detector when the surface approaches the intra-cochlear canal wall or basilar membrane. In both types of sensor, the change of returned light signal serves as an indication of imminent contact and provides a feedback or an alarm for the surgeon who performs the insertion procedure.

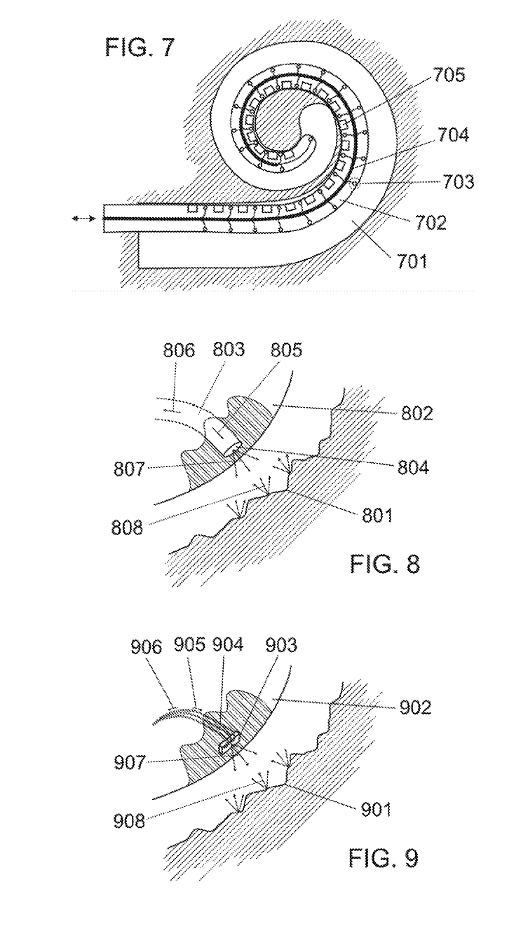

The present invention provides an integration of a cochlear implant and an optical proximity sensor in order to enable atraumatic cochlear implant surgery. The cochlear implant may be fabricated with a soft polymer, polydimethylsiloxane (PDMS), which encapsulates the electrode array and wirings that stimulate the inner-ear sensory neurons with the electrical signals converted from external acoustic vibrations. As illustrated in FIG. 7 the present invention incorporates proximity sensors 703 and necessary means for transport 704 of sensing energy and signals in the middle of such a device. The means of transport 704 provides sensing energy in the form of either illumination light or electricity to the proximity sensor 703 at selected sensing sites along the surface of the implant body 702. The proximity sensor 703 directs a light beam, either delivered by the transport pathway or generated locally, to the internal structure of the inner ear, typically the wall of the scala tympani 701, including the basilar membrane. The scattered light is either collected in the form of a light signal or converted locally into an electrical signal, and is delivered by the transport means 704 to the outside world. The measured intensity of the scattered light indicates the distance between the tip of the proximity sensor to the canal wall. This signal will be analyzed. As a result, a warning may be produced and given to the surgeon if the cells in the cochlea are likely to be compromised by the ongoing insertion trajectory. In one embodiment, the feedback signal may be converted into a useable form (for example, an audible alarm) that will prompt the surgeon to adjust the insertion angle. The proximity sensors 703 may be sacrificed, or in other words, remain inside the implant without the need for extraction, after the insertion procedure.

In one embodiment illustrated in FIG. 8, at least one waveguide structure 803 provides the means of transport of the illuminating light 805 and signal light 806. The distal tip of the waveguide 804 located at a specific position on the surface 802 of the implant body with the electrode array which forms the proximity sensor. The light 807 emitted from each fiber is scattered by the inner structure 801 of the cochlea. A portion of the backscattered light 808 from the nearby tissue is captured by the fibers at the distal tip 804, providing the proximity signal at that specific location. In another embodiment, multiple waveguides provide the means of transport and sensors at a plurality of predetermined positions on the implant surface. The waveguides are bundled along the center of the implant and are illuminated with, but not limited to, a light emitting diode or a laser diode at the proximal end. A distinct wavelength for each channel can also be used to enhance the ability of depth profile measurements. The wavelength diversity allows us to determine the strength of the coupling between any two fibers. Since the coupling is induced by the light scattering from the surrounding tissue, it is possible to use the coupling measurements to estimate the 3D shape of the cochlea surface.

The preferable method to fabricate the waveguides is direct laser-writing through two-photon or ultraviolet laser absorption in the PDMS implant body. When exposed to an intense focused near infrared or ultraviolet laser light, PDMS undergoes a crosslinking process that results in a change of refractive index at the exposed spot. The resulting refractive index contrast ranges from 0.001 to 0.01 depending on the exposure dosage. Direct laser-writing is capable of producing arbitrary three-dimensional waveguides. The relatively low index contrast, however, requires a larger waveguide size and inter-waveguide distance. Consequently the total number of waveguides and sensors that can be packed in one implant is lower compared with other methods.

The waveguides can also be fabricated by embedding thin silica or polydiphenylsiloxane (PDPS) fibers. The refractive indices of silica and PDPS, 1.47 and 1.5 respectively, are much higher than that of PDMS, 1.41, which serves as the cladding in such a scheme. The index contrast of 0.06-0.09 enables the use of very thin fibers of as small as 1 μm diameter. The fibers can also be packed close to each other without inter-fiber coupling of light. In addition, the use of very thin fibers also minimizes any potential change in the mechanical properties of the implant.

In another embodiment, the proximity sensor is constructed with only a single waveguide in the form of a single-mode optical fiber. The single-mode fiber, embedded in the center of the implant, is pre-inscribed with Bragg gratings at distinct sensing locations reflecting a specific spectral content from a broadband source into the perpendicular directions i.e. out of the fiber length. Multiple gratings can be superimposed at the same location to cover several directions in the perpendicular plane. The proximal end of the fiber is illuminated with a broadband light, and light of a specific wavelength is directed toward the canal wall by the Bragg grating. The scattered light from the nearby tissue couples back into the fiber through the same grating, the overall strength of which serves the proximity indicator. At the proximal end, a spectral analyzer separates the channels of different colors and provides the proximity signal at all sensing locations simultaneously.

In yet another embodiment illustrated in FIG. 9, the means of transport for illumination energy 905 and sensor signal 906 is provided by at least one pair of metal wires 904, which is connected to at least one proximity sensor fabricated on a small semiconductor chip 903 that integrates at least one light source such as emitting diode or laser diode and a photodetector. The light 907 emitted from the light source on the semiconductor chip 903 located on at a specific position beneath the surface 902 of the implant body with the electrode array is scattered by the inner structure 901 of the cochlea. A portion of the backscattered light 908 from the nearby tissue is detected by the photodetector fabricated on the semiconductor chip 903, providing the proximity signal at that specific location. Additional electronics, such as amplifiers and analog-to-digital converters, can also be integrated on the same chip. The metal wiring is fully compatible with the fabrication process of the electrode array. In another embodiment, the electronic proximity sensor is integrated into each electrode pad and share the same wire of the electrode. The electronic proximity sensor does not require additional fabrication steps besides those for the electrode array.

It is to be understood that, by systematic, strategic, and sufficient placement of said sensors, the implant is capable of measuring the surface profile of the cochlea canal given that the shape of the implant is known. This profile information is often highly valuable for optimal treatment of diseases in the inner ear.

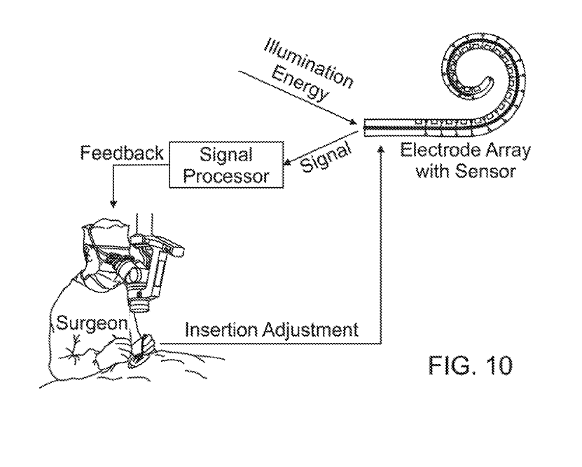

In a practical application, the proximity sensors in the implant transmits signals that can be processed and converted into a form of audio or visual feedback to the surgeon that performs the surgical insertion (FIG. 10). The surgeon then adjusts the insertion angle and force in order to avoid any damage to the organ of Corti.

1-16. (canceled)

17. A cochlear implant device comprising:
an implant body being delimited by an implant surface;
an electrode array; and
a proximity sensor as an integral part of the implant body,
wherein the proximity sensor is configured to provide a distance information between the implant surface and a cochlear intra-canal structure by measurement of a light intensity reflected from the cochlear intra-canal structure.

18. The cochlear implant device of claim 17,
wherein the proximity sensor includes a first optical waveguide and a photodetector, the first optical waveguide configured to deliver light from a source to a specific location of the implant surface, the first optical waveguide connected to the source,
wherein the light is configured to impinge the cochlea intra-canal structure and to be scattered back to the implant surface,
wherein the proximity sensor includes a second optical waveguide configured to collect the scattered light and deliver the scattered light to the photodetector, and
a signal at an output of the photodetector configured to provide the distance information.

19. The cochlear implant device of claim 18, wherein the first optical waveguide connected to the source and the second optical waveguide connected to the detector are a same optical waveguide.

20. The cochlear implant device of claim 18, wherein the first and the second optical waveguide include a thin optical fiber.

21. The cochlear implant device of claim 17, wherein the proximity sensor includes a plurality of optical waveguides,
wherein at least one of the plurality of waveguides is configured to deliver light of a wavelength such that the delivered light impinges and is scattered from the cochlear intra-canal structure, and
wherein the scattered light is collected by the plurality of waveguides,
the implant device further comprising:
a processing device configured to process an optical power collected from the plurality of waveguides to retrieve the distance information.

22. The cochlear implant device of claim 21, wherein the plurality of waveguides are configured to form a canal surface profilometer.

23. The cochlear implant device of claim 17, wherein the proximity sensor includes at least one optical waveguide configured to deliver light of multiple wavelengths from a source to a plurality of distinct locations on the implant surface in a one-to-one mapping through fiber Bragg gratings,
wherein the delivered light is configured to impinge the cochlear intra-canal structure and is scattered back to the implant surface,
wherein the at least one waveguide is configured to collect the scattered light through the fiber Bragg gratings and to provide the collected scattered light to a spectral analyzer to determine an optical power, and
wherein the optical power provides distance information between the implant surface to the cochlear intra-canal structure at a position of the proximity sensor respective to the wavelength.

24. The cochlear implant device of claim 17, wherein the proximity sensor includes a microchip arranged at a specific position on the implant surface, the microchip including a light source and a photodetector,
wherein the microchip is configured such that light from the light source impinges the cochlear intra-canal structure and is scattered back to the implant surface, and the photodetector converts a light intensity of the scattered light into the distance information at a position of the proximity sensor.

25. The cochlear implant device of claim 22, wherein the light source includes a light emitting diode.

26. The cochlear implant device of claim 22, wherein the light source includes a laser diode.

27. The cochlear implant device of claim 17, wherein the proximity sensor includes a plurality of microchips arranged at specific positions on the implant surface,
wherein each of the microchips includes a light source and a photodetector, light from the light source configured to impinge the cochlear intra-canal structure and being scattered back to the implant surface, and
wherein the photodetector is configured to convert a light intensity of the scattered light into the distance information at the position of the proximity sensor.

28. The cochlear implant device of claim 27, wherein the light source is a light emitting diode.

29. The cochlear implant device of claim 27, wherein the light source is a laser diode.

30. The cochlear implant device of claim 27, wherein the plurality of microchips form a canal surface profilometer.

31. A cochlear implant device comprising:
an implant body having an implant surface;
an electrode array configured to stimulate a cochlear intra-canal structure;
a light source configured to emit light to the cochlear intra-canal structure;
an optical sensor configured to detect a light scattered back from the cochlear intra-canal structure; and
a signal processor configured to determine a distance between the implant surface and the cochlear intra-canal structure by measuring a light intensity of the scattered light.

32. The cochlear implant device of claim 31, further comprising:
a waveguide device configured to deliver the emitted light to the cochlear intra-canal structure and configured to deliver the scattered light to the optical sensor.

33. The cochlear implant device of claim 32, wherein the waveguide device includes a fiber Bragg grating, and wherein the signal processor is configured to determine an optical power of the scattered light to determine the distance.

34. The cochlear implant device of claim 31, wherein the light source and the optical sensor are integral parts of the implant body.
